(19) United States
(12) Patent Application Publication
(10) Pub. No.: US 2006/0251753 A1
(43) Pub. Date: Nov. 9, 2006

(54) Ellagic acid food supplement prepared from pomegranate seed

(76) Inventor: Ahmad Alkayali, Lake Forest, CA (US)

Correspondence Address: Law Office of Terry L. Miller, 24832 Via San Fernando, Mission Viejo, CA, 92692, US

(21) Appl. No.: 11/125,596

(22) Filed: May 9, 2005

(51) Int. Cl. A61K 36/185; A61K 31/366

(52) U.S. Cl. 424/776; 514/453

(57) ABSTRACT

A food supplement material, and method of its preparation, containing ellagic acid (i.e., in the form of bio-available ellagitannins), derived from pomegranate plant materials. Most preferably, the food supplement is extracted from pomegranate seed, and contains from about 40% to about 90% ellagic acid (ellagitannins), and is presented for human oral administration as a dry powder contained in gelatin capsules for daily consumption.

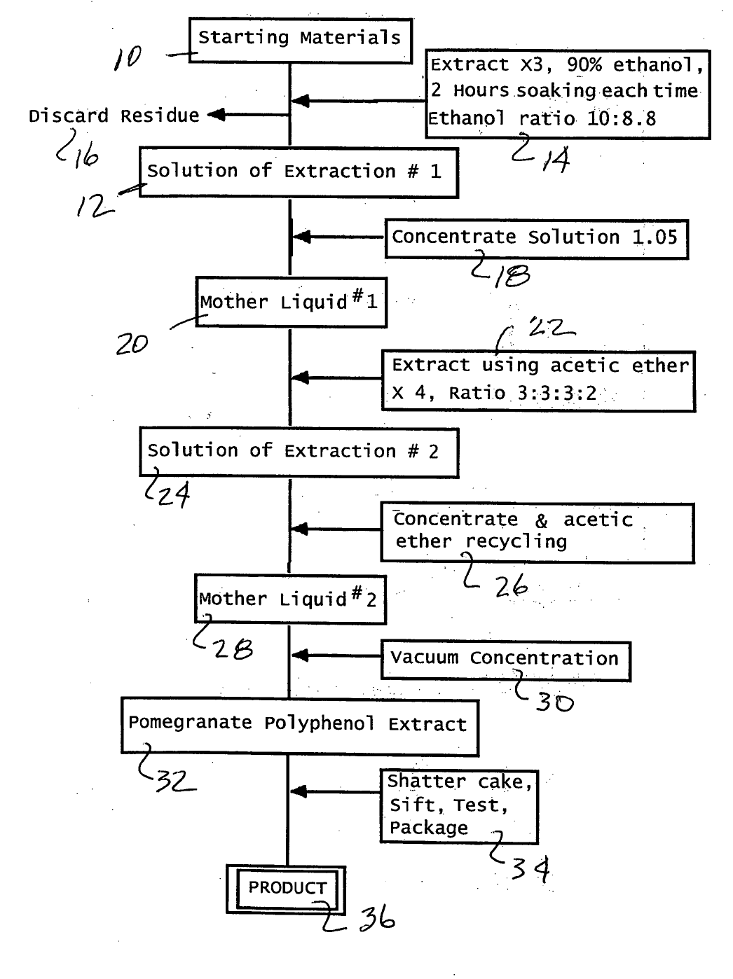

### FIELD OF THE INVENTION

The present invention relates to a method of preparing ellagic acid (in the form of pomegranate polyphenol extract) from pomegranate seed. The product material is preferably prepared in the form of a fine powder. And, the invention further relates to the beneficial use of the product material as a human nutritional or food supplement.

### BACKGROUND OF THE INVENTION

Ellagic acid is a naturally-occurring phytochemical pesticide found in a variety plant species. This biochemical serves a number of possible functions in these plants, such as regulating plant growth and seed germination, and protecting the plant from microbial infections. Ellagic acid may also protect plants from cancer-inducing chemicals, heavy metal poisoning, and predation by insects and insect larvae. Since the early 1990s, researchers have been discovering that ellagic acid offers health benefits to humans as well.

Early evidence shows that ellagic acid acts as a scavenger to “bind” or chemically engage cancer-causing chemicals, making them inactive. In addition, ellagic acid is a fused bi-nuclear coumarin derivative, which appears to prevent the binding of carcinogens to DNA, and also appears to reduce the incidence of cancer in cultured human cells exposed to carcinogens.

Although ellagic acid is the bioactive agent that offers protection, the phytochemical is generally ingested in the form of another biochemical called ellagitannin. Plants produce ellagic acid and glucose that combine to form ellagitannins, which are water-soluble compounds that are bio-available, or easier for animals to absorb in their diets.

For example, the Hollings Cancer Institute at the University of South Carolina conducted a double blind study on a large group of 500 cervical cancer patients. Nine years of study have shown that ellagic acid causes G-arrest within 48 hours (inhibiting and stopping mitosis-cancer cell division); and leads to apoptosis (normal cell death) within 72 hours, for breast, pancreas, esophageal, skin, colon and prostate cancer cells. Clinical tests also showed that ellagic acid prevents the destruction of the p53 gene by cancer cells. Additional studies suggest that one of the mechanisms by which ellagic acid inhibits mutagenesis and carcinogenesis is by forming adducts with DNA, thus masking binding sites to be occupied by the mutagen or carcinogen.

Further a publication entitled, “The American Cancer Society's Guide to Complementary and Alternative Cancer Methods” has documented that ellagic acid is a very promising natural supplement, because it causes apoptosis (cell death) of cancer cells in the lab tests, with no change to normal healthy cells. Moreover, is appears that healthy cells have a normal life cycle of approximately 120 days before they die. This process is called apoptosis (natural cell death). The body replaces these dying cells with healthy cells. Conversely, cancer cells do not die. They multiply by division, making 2 cancer cells, then 4, 8, 16, 32 and so on. In lab tests, ellagic acid has shown positive results in causing cancer cells to go through the normal apoptosis process without damaging healthy cells. Chemotherapy, radiation, and most conventional treatments cause the death of cancer cells as well as many healthy cells. This lack of discrimination in the conventional treatments for cancer can possibly destroy the immune system in the process.

Accordingly, it is to be appreciated that there is a constant need for compositions capable of preventing disease and promoting health and repair of damaged tissues. The present invention addresses this need.

### SUMMARY OF THE INVENTION

It is well understood that ellagic acid can be found in different foods. However, the bio-availability of the ellagic acid (or ellagitannins) from various plant sources is not well established. It is believed that pomegranate, and particularly, pomegranate seeds, offers an advantageous source of bio-available ellagic acid for use as a food supplement.

The present invention offers a method of extracting ellagic acid (or ellagitannins) from pomegranate seed, and for human oral administration as a nutritional supplement, therapeutic or prophylactic agent.

Accordingly, the present invention teaches a method of providing a food supplement including ellagic acid (or a polyphenol extract, or ellagitannin), and for using this food supplement for beneficial human consumption.

### BRIEF DESCRIPTION OF THE DRAWING

The single drawing Figure is a schematic diagram of a process (series of method steps) for preparing ellagic acid powder for use as a food supplement.

### DETAILED DESCRIPTION OF THE PREFERRED EMBODIMENTS

Considering now the drawing Figure, it is seen that a method of providing ellagic acid as a food supplement according to this invention begins with a stating material, indicated at step 10. Most preferably, the starting material 10 includes or consists essentially of ground or crushed pomegranate seed. As an alternative, the husk or meat of the pomegranate fruit may be included in the starting material. Still alternatively, the starting material may include pomegranate leaves.

In order to produce a first solution of extraction 12, from the starting materials 10, these starting materials are soaked (indicated as step 14 on the Figure) a minimum of 2 hours in an ethanol solution, which may comprise as much as about 90% ethanol. As is noted on the drawing Figure, the solution used to conduct the soaking steps may alternatively be in a ratio of about 10:8.8, which is about 55% ethanol, although the invention is not limited to this precise ratio. The soaking step is preferably repeated three times, with the solution being drained off and saved (possibly accompanied by mild pressing of the residue to remove excess moisture) at the conclusion of each soaking interval. The saved solutions (i.e., from the three soaking steps) are combined and are indicated as solution of extraction #1 on the Figure. As is indicated at 16 on the Figure, the residue (i.e., solid residue) is discarded.

Next, the solution of extraction #1 (i.e., indicated at 12 on the Figure) is concentrated by a ratio of about 1:05 (i.e., about a 20:1 reduction in volume) (indicated at 18 on the Figure) to produce a so-called mother liquid #1 (indicated at 20 on the Figure). This mother liquid #1 is subjected to an extraction (indicated at 22 on the Figure) utilizing acetic ether. The extraction is repeated four (4) times, using a ratio of 3:3:3:2. Consequently, a solution of extraction #2 (indicated at 24 on the Figure) is produced. This solution of extraction #2 is concentrated and acetic ether is recycled (indicated at step 26 on the Figure).

The steps recited above result in a so-called mother liquid #2 (indicated at 28 on the Figure). This mother liquid #2 is next subjected to vacuum concentration (or vacuum drying), indicated at 30 on the Figure, to produce an intermediate product 32. This intermediate product 32 is pomegranate polyphenol extract in a solid, dry cake form. The cake form of intermediate product 32 is most preferably subjected to cake shattering (i.e., breaking, milling, and/or possibly grinding) of the cake form intermediate product, to form a powder. The powder is sifted for size, is tested, and is then packaged (all indicated at 34 on the Figure), to produce the product 36. It is to be noted that dependent upon the particulars of the starting materials 10, and of the process used according to the Figure and the description above, the ellagic acid present in the product 36 may range from about 40% to about 90%.

Most desirably, the dry product 36 is packaged into soft gelatin capsules for oral consumption by humans. That is, the capsules may contain, for example, about 200 mg of ellagic acid in a bio-available form, which may be consumed by humans as a food supplement. This food supplement may have, when orally administrated as a nutritional supplement, therapeutic or prophylactic effects. Further, formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil.

Further, aqueous or other liquid suspensions may contain the ellagic acid of the invention in admixture with excipients suitable for the manufacture of aqueous suspensions. Such excipients include suspending agents, dispersing or wetting agents, one or more preservatives, one or more coloring agents, one or more flavoring agents and one or more sweetening agents such as sucrose or saccharin. Alternatively, oil suspensions may be formulated by suspending the active ingredient in a vegetable oil, such as arachis oil, olive oil, sesame oil or coconut oil, or in a mineral oil such as liquid paraffin. The oil suspension may contain a thickening agent, such as beeswax, hard paraffin or cetyl alcohol. Sweetening agents, such as those set forth above, and flavoring agents may be added to provide a palatable oral preparation. These compositions may be preserved by an added antioxidant such as ascorbic acid. So too, syrups and elixirs may be formulated with sweetening agents, such as glycerol, sorbitol or sucrose. Such formulations may also contain a demulcent, a preservative, a flavoring agent and/or a coloring agent.

For use as a nutritional supplement, prophylactic or therapeutic agent, the ellagic acid product 36, preferably packaged and presented as explained above, is orally administered in a daily dosage of between about 500 mg and 5,000 mg. More preferably, it is administered in a daily dosage of between about 2,000 mg and 4,000 mg. Most preferably, it is administered in a daily dosage of between about 2,000 and 3,000 mg per day.

The above detailed description of the invention is set forth solely to assist in understanding the invention. It is to be understood that variations of the invention, including all equivalents now known or later developed, are to be considered as falling within the scope of the invention, which is limited only by the following claims.

What is claimed is:

1. An ellagic acid (ellagitannins) containing, pomegranate-derived, material for human oral consumption as a food supplement, therapeutic or prophylactic agent, said material comprising an extract from pomegranate plant materials including pomegranate seed.

2. The material of claim 1 wherein said extract contains from about 40% to about 90% of ellagic acid (ellagitannins).

3. The material of claim 1 wherein said pomegranate plant materials includes substantially only pomegranate seed.

4. The material of claim 1 wherein said extract is presented as a dry powder packaged in gelatin capsules for human oral consumption.

5. A method of providing an ellagic acid (ellagitannins) containing material for human consumption as a food supplement, nutritional supplement, therapeutic or prophylactic, said method comprising steps of:
providing a starting material derived from the pomegranate plant;
soaking said starting material in a solution including ethanol;
saving a solution of extraction;
concentrating the solution of extraction to produce a first mother liquid;
extracting the first mother liquid using acetic ether to produce a second solution of extraction;
concentrating the second solution of extraction to produce a second mother liquid; and
concentrating the second mother liquid to produce a solid product material.

6. The method of claim 5 wherein said starting material consists essentially of pomegranate seed.

7. The method of claim 5 wherein said soaking step is repeated plural times, and each time the liquid constituent including ethanol is saved and combined to provide said first solution of extraction.

8. The method of claim 7 wherein said soaking step is repeated three times.

9. The method of claim 1 wherein said soaking step uses an ethanol solution of from about 50% ethanol to about 90% ethanol.

10. The method of claim 1 wherein said concentrating step to produce said first mother liquid from said first solution of extraction utilizes a concentration of about 20 to 1 on a volume basis.

11. The method of claim 1 wherein said first mother liquid is extracted plural times using acetic ether in order to provide said second solution of extraction.

12. The method of claim 11 wherein said plural extractions of said first mother liquid to provide said second solution of extraction is performed four times, and the four extractions respectively utilize a 3:3:3:2 ratio of mother liquid to acetic ether.

13. The method of claim 1 wherein said step of concentrating said second solution of extraction to provide said second mother liquid also includes recycling of said acetic ether.

14. The method of claim 1 wherein said step of concentrating said second mother liquid to provide said solid product material is performed using vacuum.

15. The method of claim 1 further including the steps of fracturing said solid product material to produce a powder form of product material.

16. A method of providing an ellagic acid (ellagitannins) containing food supplement, said method comprising steps of:
providing a starting material derived essentially from the seed of the pomegranate plant;
soaking said starting material plural times in a solution including ethanol;
from each soaking step saving and combining the liquid fractions to provide a solution of extraction, and disposing of the solid fraction;
concentrating the solution of extraction by a ratio of about 20:1 to produce a first mother liquid;
extracting the first mother liquid plural times using acetic ether and saving and combining the extracted solutions to produce a second solution of extraction;
concentrating the second solution of extraction to produce a second mother liquid, while providing recycling of acetic ether;
concentrating the second mother liquid to produce a solid product material; and reducing the solid product material to a powder product material.

17. The method of claim 16 wherein said starting material consists essentially only of pomegranate seed.

18. The method of claim 16 wherein said soaking step is repeated three times, and an ethanol solution of about 50% ethanol to about 90% ethanol is utilized for each soaking operation.
